(19) United States
(12) Patent Application Publication
(10) Pub. No.: US 2025/0215049 A1
(43) Pub. Date: Jul. 3, 2025

(54) PROTEIN COMPOSITIONS AND CONSUMABLE PRODUCTS THEREOF

(71) Applicant: Clara Foods Co., Daly City, CA (US)

(72) Inventor: Aniket KALE, Pleasanton, CA (US)

(73) Assignee: Clara Foods Co., Daly City, CA (US)

(21) Appl. No.: 18/799,904

(22) Filed: Aug. 9, 2024

Related U.S. Application Data
(63) Continuation of application No. PCT/US2023/012815, filed on Feb. 10, 2023.
(60) Provisional application No. 63/309,452, filed on Feb. 11, 2022.

(51) Int. Cl. C07K 1/34; C07K 1/107; C07K 14/81

(52) U.S. Cl. CPC C07K 1/34; C07K 1/107; C07K 14/8135

(57) ABSTRACT

Provided herein are methods for preparing a protein composition having a reduced quantity of a recombinant cell byproduct which provides an undesirable color to a protein composition.

### CROSS-REFERENCE TO RELATED APPLICATIONS

This application is a continuation of International Patent Application No. PCT/US2023/012815, filed Feb. 10, 2023, which claims the benefit of U.S. Provisional Patent Application No. 63/309,452, filed Feb. 11, 2022, the contents of each of which is incorporated herein by reference in its entirety.

### BACKGROUND

Proteins are important dietary nutrients. They can serve as a fuel source or as sources of amino acids, including the essential amino acids that cannot be synthesized by the body. The daily recommended intake of protein for healthy adults is 10% to 35% of a person's total calorie needs, and currently the majority of protein intake for most humans is from animal-based sources. In addition, athletes and bodybuilders may rely upon increased protein consumption to build muscle mass and improve performance. With the world population growth and the coinciding growth in global food demand, there is a need to provide alternative sustainable, non-animal-based sources of proteins as useful source of protein for daily diet, dietary supplementation and sports nutrition.

### SUMMARY

The present disclosure provides methods for producing consumable recombinant proteins that are substantially free from herein-disclosed undesired byproducts.

An aspect of the present disclosure is a method for preparing a protein composition having a reduced quantity of a recombinant cell byproduct. In some embodiments, the recombinant cell byproduct provides an undesirable color to the protein composition. In some embodiments, the method comprises steps of: obtaining a composition having an undesirable color and comprising a recombinant protein and a recombinant cell byproduct; processing the composition having the undesirable color under conditions that oxidizes the recombinant cell byproduct or under conditions that separate the recombinant protein and the recombinant cell byproduct; and collecting a composition comprising the oxidized recombinant cell byproduct or collecting the separated recombinant protein, thereby obtaining a protein composition having a reduced quantity of the recombinant cell byproduct which provides an undesirable color.

In some embodiments, the step of processing the composition comprises use of an absorbent or flocculant which captures, traps, or isolates the recombinant cell byproduct. In some embodiments, the separated recombinant protein comprises use of gravity directed to the absorbent or the flocculant that has captured, trapped, or isolated the recombinant cell byproduct, comprise use of a filter that restricts passage of the absorbent or the flocculant that has captured, trapped, or isolated the recombinant cell byproduct and/or comprises use of centrifugation directed to the absorbent or the flocculant that has captured, trapped, or isolated the recombinant cell byproduct. In some embodiments, when gravity or centrifugation is used, the collecting the separated recombinant protein comprises decanting or aspirating a protein composition having a reduced quantity of the recombinant cell byproduct which provides an undesirable color. In some embodiments, when a filter is used, the collecting the separated recombinant protein comprises allowing the protein composition having a reduced quantity of the recombinant cell byproduct which provides an undesirable color to flow through the filter. In some embodiments, the absorbent or the flocculant comprises one or more of Bentonite, Activated Carbon, Diaion HPA25L, Relisorb SP400, EpMinerals FP1, Florisil, Celite Standard Supercel, Dicalite, Harborlite 800, Celite 545, Chitosan 85% Deacylated, Ultrapure Diatomaceous Earth, XAD4. In some embodiments, the absorbent or the flocculant comprises two or more of Bentonite, Activated Carbon, Diaion HPA25L, Relisorb SP400, EpMinerals FP1, Florisil, Celite Standard Supercel, Dicalite, Harborlite 800, Celite 545, Chitosan 85% Deacylated, Ultrapure Diatomaceous Earth, XAD4. In some embodiments, the absorbent or the flocculant comprises bentonite. In some embodiments, the absorbent or flocculant is activated carbon. In some embodiments, the spectrophotometric absorbance of the obtained protein composition having a reduced quantity of the recombinant cell byproduct which provides an undesirable color is less than the spectrophotometric absorbance of the composition having an undesirable color and comprising a recombinant protein and a recombinant cell byproduct.

In some embodiments, the step of processing the composition comprises use of an oxidizing agent which oxidizes the recombinant cell byproduct. In some embodiments, the step of processing the composition comprises use of an oxidizing agent which oxidizes the recombinant cell byproduct. In some embodiments, the oxidizing agent is hydrogen peroxide. In some embodiments, the step of processing the composition further comprises reducing to pH to within 2 pH of the recombinant protein's pI. In some embodiments, the step of processing the composition further comprises reducing to pH to within 1 pH of the recombinant protein's pI. In some embodiments, the step of processing the composition further comprises reducing to pH to about the pH of the recombinant protein's pI. In some embodiments, the step of processing the composition further comprises adjusting the pH of the composition to between about a pH of 3 or 4. In some embodiments, the step of processing the composition comprises an incubation period of about 9 hours. In some embodiments, the step of processing the composition comprises an incubation period of less than about 12 hours.

In some embodiments, the step of processing the composition comprises use of light which oxidizes the recombinant cell byproduct, and wherein the light is applied at an intensity and duration such that the recombinant cell byproduct is oxidized. In some embodiments, the light intensity is from 300-700 lumens.

In some embodiments, processing the composition comprises agitation.

In some embodiments, processing the composition occurs at room temperature or at a temperature less than room temperature. In some embodiments, processing the composition occurs at about 4° C.

In some embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In some embodiments, the composition comprising the recombinant protein and the recombinant cell byproduct was previously treated to remove protein molecules other than the recombinant protein.

In some embodiments, the composition comprising the recombinant protein and the recombinant cell byproduct was previously treated to remove small non-protein molecules. In some embodiments, the treatment to remove small non-protein molecules comprises a diafiltration buffer. In some embodiments, the treatment to remove small non-protein molecules comprises a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic condition. In some embodiments, the protein-containing composition having a preferred pH and/or ionic conditions is further heat treated and/or dried. In some embodiments, the heat treatment and/or drying step produces a dry protein product having a reduced quantity of the off-color component.

In some embodiments, the protein product having a reduced quantity of the off-color component comprises an at least 50% reduction in off-color component quantity relative to the composition comprising a recombinant protein and a recombinant cell byproduct. In some embodiments, the protein product has an at least 75% reduction, at least 80% reduction, at least 90% reduction, or at least 95% reduction in the off-color component relative to the composition comprising a recombinant protein and a recombinant cell byproduct. In some embodiments, less than about 5%, less than about 1%, less than about 0.1%, or less than about 0.01% of the weight of the protein product comprises the off-color component.

In some embodiments, the recombinant cell that expresses and/or secretes the recombinant protein and the recombinant cell byproduct is selected from a fungal cell, such as filamentous fungus or a yeast, a bacterial cell, a plant cell, an insect cell, or a mammalian cell.

In some embodiments, the recombinant cell that expresses and/or secretes the recombinant protein and the recombinant cell byproduct is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus. In some embodiments, the recombinant cell that expresses and/or secretes the recombinant protein and the recombinant cell byproduct is a Pichia species. In some embodiments, the Pichia species is Komagataella phaffii or Komagataella pastoris.

In some embodiments, the recombinant protein is an enzyme, a nutritive protein, a food ingredient, or a food additive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material. In particular, subject matter from WO2023004172A1, WO2023004153A1, WO2022182799A1, WO2022076615A1, WO2021158817A1, WO2021034980A1, WO2021007565A1, WO2016077457A1, and PCT/US2022/038074 is incorporated herein by reference in its entirety.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 is an illustrative method for producing a composition comprising a recombinant protein and a recombinant cell byproduct and separate the recombinant protein and the recombinant cell byproduct.

FIG. 2 shows pictures of samples pretreatment and post treatment with no H2O2.

FIG. 3 shows pictures of samples with 1% v/v H2O2 with varying hours of treatment.

FIG. 4 shows pictures of samples showing the effect of pH and H2O2. Each tube is labeled with a three-digit number. The first digit indicates the pH, the second digit indicates the % H2O2, and the third digit indicates the incubation time (in hours).

FIG. 5 shows pictures of select samples of the treatment showing the effect of pH and pH with H2O2. Tubes are labeled according to the three-digit nomenclature described above for FIG. 4.

FIG. 6A and FIG. 6B show the absorption spectra of the supernatant in duplicate samples containing the one of the adsorbents and flocculants indicated on the right of each plot. The lower the absorbance value, the more de-colored the product is.

FIG. 7A and FIG. 7B. An illustrative photo demonstrating the color of the initial protein concentrate (FIG. 7A) and an illustrative photo of supernatant after bentonite has been added to a 10% w/v solution of the protein concentrate (FIG. 7B).

FIG. 8 depicts a schematic of a device for photochemical oxidation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are methods of purifying proteins, in particular removing impurities bound to proteins of interest. In some aspects, the present disclosure provides methods and systems to purify recombinantly expressed proteins.

It has been discovered that when recombinant proteins are produced by fermenting yeast cells, such as Pichia, the recombinant cells likewise produces recombinant cell byproducts. The presence of recombinant cell byproducts in a protein product or consumable composition may have a non-preferred or undesirable color and/or taste. Accordingly, methods for processing a composition comprising a recombinant protein and a recombinant cell byproduct to separate the recombinant protein and the recombinant cell byproduct, i.e., an off-color component, is needed.

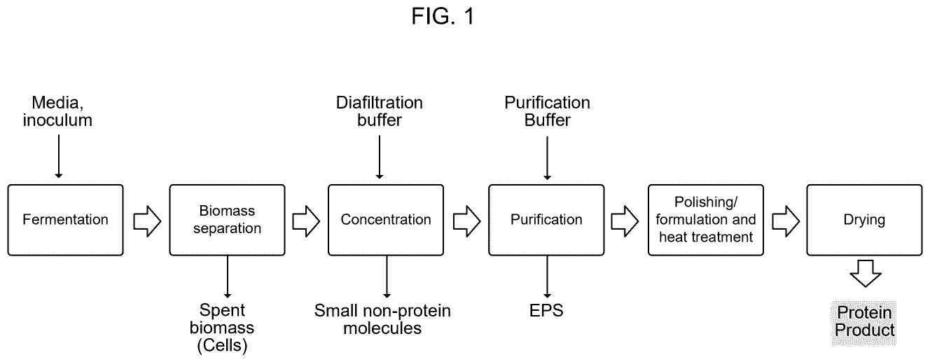

A generic method for producing a composition comprising a recombinant protein and a recombinant cell byproduct and separate the recombinant protein and the recombinant cell byproduct i.e., an off-color/flavor component, is shown in FIG. 1.

Examples of proteins that can benefit from removal of impurities include but are not limited to corn protein (prolamin zein), berberine protein, ovomucoid, etc.

Examples of impurities that can be removed using the present disclosure include but are not limited to carotenoids, folate, flavin adenine dinucleotide (FAD) molecules, etc.

The methods described herein can be particularly useful in conditions where typical methods such as ion exchange chromatography or diafiltration do not work as efficiently as desired. The methods described herein may be combined with typical methods of protein purification such as ion exchange chromatography or diafiltration.

### Oxidizing Solvent-Based Purification

An aspect of the present disclosure is a method for preparing a protein composition having a reduced quantity of a recombinant cell byproduct. In some embodiments, the recombinant cell byproduct provides an undesirable color to a protein composition. The method comprises steps of: obtaining a composition having an undesirable color and comprising a recombinant protein and a recombinant cell byproduct; processing the composition having the undesirable color under conditions that oxidizes the recombinant cell byproduct or under conditions that separate the recombinant protein and the recombinant cell byproduct; and collecting a composition comprising the oxidized recombinant cell byproduct or collecting the separated recombinant protein, thereby obtaining a protein composition having a reduced quantity of the recombinant cell byproduct which provides an undesirable color. In some embodiments, the recombinant cell byproduct is an off-color component. In some embodiments, the step of processing the composition comprises use of an oxidizing solvent (e.g., hydrogen peroxide) which separates the recombinant protein and the recombinant cell byproduct.

An illustrative method for producing a composition comprising a recombinant protein and a recombinant cell byproduct and separate the recombinant protein and the recombinant cell byproduct is shown in FIG. 1.

In some embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In some embodiments, the composition comprising the recombinant protein and the recombinant cell byproduct was previously treated to remove protein molecules other than the recombinant protein.

In various embodiments, the composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic condition. In some cases, protein-containing composition having a preferred pH and/or ionic conditions is further heat treated and/or dried. The heat treatment and/or drying step may produce a dry protein product having a reduced quantity of the off-color component.

In various embodiments, the protein product having a reduced quantity of the off-color component comprises an at least 50% reduction in off-color component quantity relative to the composition comprising a recombinant protein and a recombinant cell byproduct. In some cases, the protein product has an at least 75% reduction, at least 80% reduction, at least 90% reduction, or at least 95% reduction in the off-color component relative to the composition comprising a recombinant protein and a recombinant cell byproduct.

In embodiments, less than about 5%, less than about 1%, less than about 0.1%, or less than about 0.01% of the weight of the protein product comprises the off-color component. In some cases, the off-color component in the protein product is virtually undetectable to a standard consumer.

The protein of interest, may be treated chemically or enzymatically before it is purified for use in a consumable composition or protein mixture. Such treatments may be performed to reduce impurities in a protein of interest composition. Such treatments may be performed to improve the sensory attributes of the protein of interest. Treatments may include but are not limited to purification steps, filtration, chemical treatments, and enzymatic treatments.

In some cases, protein of interest may be treated with oxidizing agent or an oxygen-generating agent to modify components of the composition, such as impurities. The oxidizing agent or oxygen-generating agent may comprise hydrogen peroxide, sodium percarbonate, activated chlorine dioxide, bubbled oxygen or ozone. The treatment may improve the solubility and clarity of an protein of interest. The treatment may reduce the odor of an protein of interest. The treatment may neutralize the color of an protein of interest; for instance, the protein of interest composition may lose color after a treatment, e.g., to a less intense/lighter coloration. In embodiments, the color may change form greenish to yellowish and/or from yellowish to essentially colorless.

In some examples, protein of interest may be treated with an oxidizing agent or an oxygen-generating agent, e.g., hydrogen peroxide or sodium percarbonate, before it is purified for use in a consumable composition. A culture medium comprising secreted or isolated protein of interest may be treated with an oxygen-generating agent, e.g., hydrogen peroxide or sodium percarbonate. Using hydrogen peroxide as an example, a hydrogen peroxide treatment may be followed by one or more wash steps and/or filtration steps to remove hydrogen peroxide from the resulting protein of interest. Such steps may be performed following treatments with other oxygen-generating agents, e.g., sodium percarbonate.

In some cases, the concentration of hydrogen peroxide used for treating protein of interest may be from 1% to 20%. The concentration of hydrogen peroxide used for treating protein of interest may be at least 1% weight per total weight (w/w) and/or weight per total volume (w/v). The concentration of hydrogen peroxide used for treating protein of interest may be at most 20% w/w or w/v. The concentration of hydrogen peroxide used for treating protein of interest may be 1% to 2%, 1% to 5%, 1% to 7%, 1% to 10%, 1% to 12%, 1% to 15%, 1% to 17%, 1% to 20%, 2% to 5%, 2% to 7%, 2% to 10%, 2% to 12%, 2% to 15%, 2% to 17%, 2% to 20%, 5% to 7%, 5% to 10%, 5% to 12%, 5% to 15%, 5% to 17%, 5% to 20%, 7% to 10%, 7% to 12%, 7% to 15%, 7% to 17%, 7% to 20%, 10% to 12%, 10% to 15%, 10% to 17%, 10% to 20%, 12% to 15%, 12% to 17%, 12% to 20%, 15% to 17%, 15% to 20%, or 17% to 20% w/w or w/v. The concentration of hydrogen peroxide used for treating protein of interest may be about 1%, 2%, 5%, 7%, 10%, 12%, 15%, 17%, or 20% w/w or w/v. The concentration of hydrogen peroxide used for treating protein of interest may be at least 1%, 2%, 5%, 7%, 10%, 12%, 15% or 17% w/w or w/v. The concentration of hydrogen peroxide used for treating protein of interest may be at most 2%, 5%, 7%, 10%, 12%, 15%, 17%, or 20% w/w or w/v.

protein of interest may be treated with hydrogen peroxide for a limited duration of time. For instance, protein of interest may be exposed to hydrogen peroxide for at least 1 hour, 2 hours, 3 hours, 5 hours, 7 hours, 10 hours, 12 hours, 15 hours, 17 hours, 20 hours, 22 hours, 24 hours, 26 hours, 28 hours, 30 hours, 34 hours, 36 hours, 40 hours, 44 hours or 48 hours. Hydrogen peroxide may be added to the protein of interest culture media throughout the culturing process.

protein of interest may be treated with hydrogen peroxide at a pH of about 3 to 6. protein of interest may be treated with hydrogen peroxide at a pH of about 3, 3.2, 3.4, 3.6, 3.8, 4, 4.1, 4.2, 4.4, 4.6, 4.8, 5, 5.2, 5.4, 5.6, 5.8 or 6. protein of interest may treated with hydrogen peroxide at a pH of at least 3, 3.2, 3.4, 3.6, 3.8, 4, 4.1, 4.2, 4.4, 4.6, 4.8, 5, 5.2, 5.4, 5.6 or 5.8. protein of interest may treated with hydrogen peroxide at a pH of at most 3.2, 3.4, 3.6, 3.8, 4, 4.1, 4.2, 4.4, 4.6, 4.8, 5, 5.2, 5.4, 5.6, 5.8 or 6.

protein of interest may be filtered before treatment with an oxygen-generating agent. In some cases, protein of interest may be filtered before and after treatment with an oxygen-generating agent.

### Purification Using Absorbents and Flocculants

An aspect of the present disclosure is a method for preparing a protein composition having a reduced quantity of a recombinant cell byproduct. The method comprises steps of: obtaining a composition comprising a recombinant protein and a recombinant cell byproduct which provides an undesirable color to a protein composition; processing the composition under conditions that separate the recombinant protein and the recombinant cell byproduct; collecting the separated recombinant protein, thereby obtaining a protein composition having a reduced quantity of the recombinant cell byproduct which provides an undesirable color. In this method the recombinant cell byproduct is an off-color component. In various embodiments, the step of processing the composition processing the composition comprises use of an absorbent or flocculant which captures, traps, or isolates the recombinant cell byproduct. In some embodiments, the absorbent or the flocculant does not substantially capture, trap, or isolate the recombinant protein

A illustrative method for producing a composition comprising a recombinant protein and a recombinant cell byproduct and separate the recombinant protein and the recombinant cell byproduct is shown in FIG. 1.

In some embodiments, the collecting the separated recombinant protein comprises use of gravity directed to the absorbent or the flocculant that has captured, trapped, or isolated the recombinant cell byproduct, comprise use of a filter that restricts passage of the absorbent or the flocculant that has captured, trapped, or isolated the recombinant cell byproduct and/or comprises use of centrifugation directed to the absorbent or the flocculant that has captured, trapped, or isolated the recombinant cell byproduct.

In some embodiments, gravity or centrifugation is used, the collecting the separated recombinant protein comprises decanting or aspirating a protein composition having a reduced quantity of the recombinant cell byproduct which provides an undesirable color.

In embodiments when a filter is used, the collecting the separated recombinant protein comprises allowing the protein composition having a reduced quantity of the recombinant cell byproduct which provides an undesirable color to flow through the filter.

In some embodiments, the absorbent or the flocculant comprises one or more of Bentonite, Activated Carbon, Diaion HPA25L, Relisorb SP400, EpMinerals FP1, Florisil, Celite Standard Supercel, Dicalite, Harborlite 800, Celite 545, Chitosan 85% Deacylated, Ultrapure Diatomaceous Earth, XAD4. In some embodiments, the absorbent or the flocculant comprises two or more of Bentonite, Activated Carbon, Diaion HPA25L, Relisorb SP400, EpMinerals FP1, Florisil, Celite Standard Supercel, Dicalite, Harborlite 800, Celite 545, Chitosan 85% Deacylated, Ultrapure Diatomaceous Earth, XAD4. In some embodiments, the absorbent or the flocculant comprises bentonite. In some embodiments, the absorbent or flocculant is activated carbon.

In some embodiments, the spectrophotometric absorbance of the obtained protein composition having a reduced quantity of the recombinant cell byproduct which provides an undesirable color is less than the spectrophotometric absorbance of the composition having an undesirable color and comprising a recombinant protein and a recombinant cell byproduct.

In embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In various embodiments, the composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic conditions. In some cases, protein-containing composition having a preferred pH and/or ionic conditions is further light treated and/or dried. The light treatment and/or drying step may produce a dry protein product having a reduced quantity of the off-color component.

In various embodiments, the protein product having a reduced quantity of the off-color component comprises an at least 50% reduction in off-color component quantity relative to the composition comprising a recombinant protein and a recombinant cell byproduct. In some cases, the protein product has an at least 75% reduction, at least 80% reduction, at least 90% reduction, or at least 95% reduction in the off-color component relative to the composition comprising a recombinant protein and a recombinant cell byproduct.

In embodiments, less than about 5%, less than about 1%, less than about 0.1%, or less than about 0.01% of the weight of the protein product comprises the off-color component. In some cases, the off-color component in the protein product is virtually undetectable to a standard consumer.

### Chemical Oxidation-Based Purification

An aspect of the present disclosure is a method for preparing a protein composition having a reduced quantity of a recombinant cell byproduct. The method comprises steps of: obtaining a composition comprising a recombinant protein and a recombinant cell byproduct which provides an undesirable color to a protein composition; processing the composition under conditions that separate the recombinant protein and the recombinant cell byproduct; collecting the separated recombinant protein, thereby obtaining a protein composition having a reduced quantity of the recombinant cell byproduct which provides an undesirable color. In this method the recombinant cell byproduct is an off-color component. In various embodiments, the step of processing the composition comprises use of an oxidizing agent which oxidizes the recombinant cell byproduct. In some embodiments, the oxidizing agent is hydrogen peroxide.

An illustrative method for producing a composition comprising a recombinant protein and a recombinant cell byproduct and separate the recombinant protein and the recombinant cell byproduct is shown in FIG. 1.

In some embodiments, the step of processing the composition further comprises reducing to pH to within 2 pH of the recombinant protein's pI. In some embodiments, the step of processing the composition further comprises reducing to pH to within 1 pH of the recombinant protein's pI. In some embodiments, the step of processing the composition further comprises reducing to pH to about the pH of the recombinant protein's pI.

In some embodiments, the step of processing the composition further comprises adjusting the pH of the composition to between about a pH of 3 or 4.

In some embodiments, the step of processing the composition comprises an incubation period between about 9 hours and about 12 hours. In some embodiments, the incubation period is about 9 hours. In some embodiments, the incubation period is less than about 12 hours.

In embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In various embodiments, the composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic conditions. In some cases, protein-containing composition having a preferred pH and/or ionic conditions is further light treated and/or dried. The light treatment and/or drying step may produce a dry protein product having a reduced quantity of the off-color component.

In various embodiments, the protein product having a reduced quantity of the off-color component comprises an at least 50% reduction in off-color component quantity relative to the composition comprising a recombinant protein and a recombinant cell byproduct. In some cases, the protein product has an at least 75% reduction, at least 80% reduction, at least 90% reduction, or at least 95% reduction in the off-color component relative to the composition comprising a recombinant protein and a recombinant cell byproduct.

In embodiments, less than about 5%, less than about 1%, less than about 0.1%, or less than about 0.01% of the weight of the protein product comprises the off-color component. In some cases, the off-color component in the protein product is virtually undetectable to a standard consumer.

### Light-Based Purification

An aspect of the present disclosure is a method for preparing a protein composition having a reduced quantity of a recombinant cell byproduct. The method comprises steps of: obtaining a composition comprising a recombinant protein and a recombinant cell byproduct which provides an undesirable color to a protein composition; processing the composition under conditions that separate the recombinant protein and the recombinant cell byproduct; collecting the separated recombinant protein, thereby obtaining a protein composition having a reduced quantity of the recombinant cell byproduct which provides an undesirable color. In this method the recombinant cell byproduct is an off-color component. In various embodiments, the step of processing the composition comprises use of light which separates the recombinant protein and the recombinant cell byproduct, wherein the light is applied at an intensity and duration such that the recombinant cell byproduct is oxidized and a gaseous recombinant cell byproduct is removable.

An illustrative method for producing a composition comprising a recombinant protein and a recombinant cell byproduct and separate the recombinant protein and the recombinant cell byproduct is shown in FIG. 1.

In some embodiments, the composition may be agitated while the light is applied.

In various cases, the light intensity is from 300-700 lumens.

In embodiments, the composition comprising the recombinant protein and the recombinant cell byproduct was produced by fermentation of the recombinant cell.

In some embodiments, the composition comprising the recombinant protein and the recombinant cell byproduct was previously treated to remove spent biomass including recombinant cells.

In various embodiments, the composition comprising the recombinant protein and the recombinant cell byproduct was previously treated to remove small non-protein molecules. In some cases, the treatment to remove small non-protein molecules comprises a diafiltration buffer. The treatment to remove small non-protein molecules may comprise a step that concentrates the composition comprising the recombinant protein and the recombinant cell byproduct.

In some embodiments, the method further comprises a concentration step and/or diafiltration treatment of the separated recombinant protein to produce a protein-containing composition having a preferred pH and/or ionic conditions. In some cases, protein-containing composition having a preferred pH and/or ionic conditions is further light treated and/or dried. The light treatment and/or drying step may produce a dry protein product having a reduced quantity of the off-color component.

In various embodiments, the protein product having a reduced quantity of the off-color component comprises an at least 50% reduction in off-color component quantity relative to the composition comprising a recombinant protein and a recombinant cell byproduct. In some cases, the protein product has an at least 75% reduction, at least 80% reduction, at least 90% reduction, or at least 95% reduction in the off-color component relative to the composition comprising a recombinant protein and a recombinant cell byproduct.

In embodiments, less than about 5%, less than about 1%, less than about 0.1%, or less than about 0.01% of the weight of the protein product comprises the off-color component. In some cases, the off-color component in the protein product is virtually undetectable to a standard consumer.

### Features of Methods of the Present Disclosure

In some embodiments, the recombinant cell is cell that expresses and/or secretes the recombinant protein and recombinant cell byproduct and is selected from a fungal cell, such as filamentous fungus or a yeast, a bacterial cell, a plant cell, an insect cell, or a mammalian cell.

In various embodiments, the recombinant cell type is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus. In some cases, the fungus is a Pichia species. In some cases, the Pichia species is Komagataella phaffii or Komagataella pastoris.

In embodiments, the recombinant protein is an enzyme, a nutritive protein, a food ingredient, or a food additive. In some cases, the enzyme is pepsinogen or pepsin.

In some embodiments, the protein is an egg-white protein.

In some cases, the egg-white protein is ovalbumin (OVA), ovomucoid (OVD), ovotransferrin (OVT), lysozyme (OVL), ovomucin, ovoglobulin G2, ovoglobulin G3, ovoinhibitor, ovoglycoprotein, flavoprotein, ovomacroglobulin, ovostatin, cystatin, avidin, ovalbumin related protein X, or ovalbumin related protein Y, and any combination thereof. In various cases, the egg-white protein is OVA, OVD, OVT, or OVL. In some cases, an egg-white protein has a sequence that at least 80% identical (e.g., about 85%, 90%, or 95% identical) to the egg-white protein naturally produced in a bird, e.g., a chicken, quail, turkey, turkey vulture, hummingbird, duck, ostrich, goose, gull, guineafowl, pheasant, or emu.

### Protein Products and Protein-Containing Consumable Compositions

Another aspect of the present disclosure is a protein product prepared by any herein-disclosed method.

Yet another aspect of the present disclosure is a consumable composition comprising any herein-disclosed protein product.

In various aspects, any herein-disclosed consumable composition may be used in a food product.

In an aspect, the present disclosure provides a consumable composition comprising a recombinant protein that was separated from a composition comprising a recombinant protein and a recombinant cell byproduct, wherein the recombinant cell byproduct is an off-color component.

In another aspect, the present disclosure provides method for preparing a consumable composition. The method comprising steps of: obtaining a composition comprising a recombinant protein and a recombinant cell byproduct, wherein the recombinant cell byproduct is an off-color component; processing the composition under conditions that separate the recombinant protein and the off-color component; collecting the separated recombinant protein, thereby obtaining a protein product having a reduced quantity of the off-color component; and formulating a consumable composition comprising the protein product.

In embodiments, the consumable composition further includes at least one consumable ingredient. In some cases, the consumable ingredient is a solvent, e.g., water, carbonated water, alcohol, juice, and any other commercially available drink. The consumable composition comprising the protein product and having a reduced quantity of the recombinant cell byproduct may have an improved color relative an equivalent consumable composition that does not have a reduced quantity of the recombinant cell byproduct.

In some embodiments, the consumable composition comprises one or more recombinant egg-white proteins, the consumable composition has a color similar to a consumable composition comprising natural egg-white proteins.

### Definitions

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The term “bird” includes both domesticated birds and non-domesticated birds such as wildlife and the like. Birds include, but are not limited to, poultry, fowl, waterfowl, game bird, ratite (e.g., flightless bird), chicken (Gallus gallus domesticus), quail, turkey, duck, ostrich (Struthio camelus), Somali ostrich (Struthio molybdophanes), goose, gull, guineafowl, pheasant, emu (Dromaius novaehollandiae), American rhea (Rhea americana), Darwin's rhea (Rhea pennata), and kiwi.

As used herein, the terms “consumable composition” or “consumable product” refers to a composition or product, which comprises a recombinant protein or composition comprising recombinant protein and other ingredients and may be consumed (e.g., by eating, chewing, drinking, tasting, ingesting, or swallowing). Consumable products include food products, beverage products, dietary supplements, food additives, pharmaceutical products, and hygiene products, as non-limiting examples. Food products include, but are not limited to, baked goods (e.g., cake, muffin, cookie, bread, bagel, pastry, doughnut), scramble, omelet, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings. Beverage products include, but are not limited to, soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks. Dietary supplements include multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement. A consumer of a consumable product or consumable composition is any animal, including domesticated animals (e.g., livestock) and humans.

Processing of a consumable product to form a processed consumable product may include, but is not limited to, freezing, chilling, heating, baking, roasting, broiling, boiling, blanching, packaging, canning, bleaching, enriching, drying, pressing, grinding, mixing, parcooking, cooking, proofing, marinating, cutting, slicing, dicing, crushing, shredding, chopping, shaking, coring, spiralizing, rolling, juicing, straining, filtering, kneading, whisking, beating, whipping, grating, stuffing, peeling, deseeding, smoking, curing, salting, preserving, pickling, fermenting, homogenizing, pasteurizing, sterilizing, stabilizing, blending, pureeing, fortifying, refining, hydrogenating, aging, extending shelf life, or adding enzymes.

As used herein, the term “solvent” refers to a liquid, which may be mixed with or used to dissolve a composition or one or more components of a composition such as a protein. Non-limiting examples of a solvent include water, ethanol, and isopropanol. The solvent can be potable. The solvent can be water. Non-limiting examples of water include purified water, distilled water, double distilled water, deionized water, distilled deionized water, drinking water, well water, tap water, spring water, bottled water, carbonated water, mineral water, flavored water, or any combination thereof. A solvent may be a combination of two or more distinct solvents.

Any aspect or embodiment described herein can be combined with any other aspect or embodiment as disclosed herein.

### EXAMPLES

The following examples are given for the purpose of illustrating various embodiments of the invention and are not meant to limit the present invention in any fashion. The present examples, along with the methods described herein are presently representative of preferred embodiments, are illustrative, and are not intended as limitations on the scope of the invention. Changes therein and other uses which are encompassed within the spirit of the invention as defined by the scope of the claims will occur to those skilled in the art.

### Example 1: Processing of Recombinantly Made Ovomucoid

Pichia Pastoris strain derived from the historic Phillips Petroleum strain NRRL Y-11430) was designed to express the ovomucoid protein.

The resulting strain was grown in fermentation conditions in high-density growth conditions at about pH 5 (other viable pH conditions include pH of 4-6). After about 36 hours (in some cases, fermentation may be performed from 25-65 hours) of growth under fermentation conditions, the pH was raised to about pH 6, and expression of ovomucoid was induced by the addition of methanol to the culture. The ovomucoid was isolated from the growth media of the culture. From the growth media from fermentation the liquid was centrifuged to remove cells. This was followed by filtration of the supernatant using a 0.2 um hollow fiber membrane filtration to remove host protein and cell debris. The protein solution was then concentrated using a 5 kDa ultrafiltration membrane and diafiltered to remove most of the organic and inorganic impurities. The resulting protein concentrate is typically dark olive green colored.

The protein sample was green in color and was oxidized to remove the bound FAD impurity. Treatment utilized in the process was photolytic oxidation exposing 100 mL of the ultrafiltration concentrate with or without H2O2 with white light at 480 lumen (˜40W) intensity (in some examples a light intensity from 300 to 700 lumens may be utilized) for 15 hours (in some cases, the protein sample may be treated from 10 to 20 hours in a static system or from 15 seconds to 120 seconds in a continuous system). In this example, 6 kWhr/L of incandescent light was used for instantaneous oxidation of the sample. With an LED system the same could be reduced down to 1 kWhr/L. Further with specific wavelength of the LED light this can be finetuned to 0.5 kWhr/L. With a continuous flow system and a 10 min hold per liter of sample about 50 Whr/L might be used.

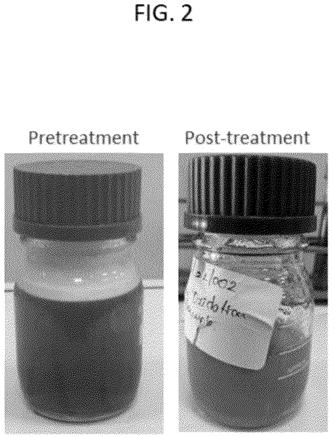

FIG. 2 shows pictures of samples pretreatment and post treatment with no H2O2.

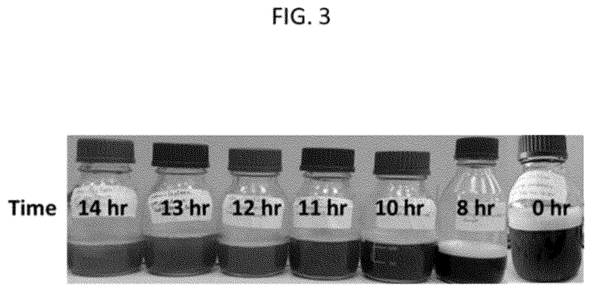

FIG. 3 shows pictures of samples with 1% v/v H2O2 with varying hours of treatment.

This technology can be implemented using a design providing maximum photons per unit volume without heat damaging the protein.

### Example 2: Decoloring Colored Protein Products Using Chemical Oxidation

A Pichia Pastoris strain was designed to express an ovalbumin protein. The strain was grown in fermentation conditions in high-density growth conditions at about pH 5. After about 36 hours of growth under fermentation conditions, the pH was raised to about pH 6, and expression of ovalbumin was induced. The ovalbumin was isolated from the growth media of the culture. From the growth media from fermentation the liquid was centrifuged to remove cells. This was followed by filtration of the supernatant using a 0.2 μm hollow fiber membrane filtration to remove host protein and cell debris. The protein solution was then concentrated using a 5 kDa ultrafiltration membrane to over 30 g/L protein and diafiltered extensively to remove most of the organic and inorganic impurities. The resulting protein ultrafiltration concentrate was typically dark olive green colored.

A strong acid, 85% phosphoric acid, was added to the dark olive green colored resulting protein concentrate to reduce the pH to 3.5±0.5, followed by addition of a strong oxidizer, e.g., hydrogen peroxide, to make the concentration of the solution about 5% v/v. The solution was then stirred for about 12 hours followed by extensive diafiltration to remove any oxidation degradation products and remanent peroxide in the solution. The final concentration was then sterile filtered and dried.

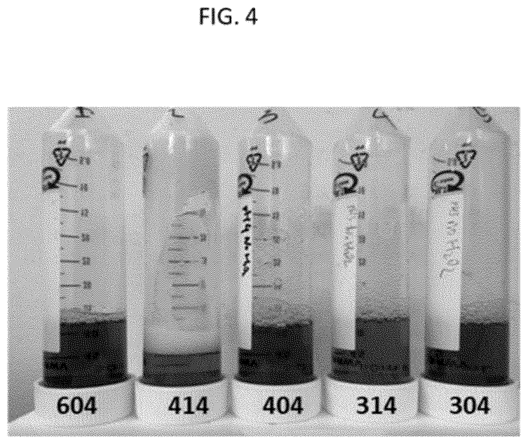

FIG. 4 shows illustrative samples of the treatment showing the effects of altered pH with or without a strong chemical oxidizer. The three-digit code describes the conditions of the de-coloration treatment: The first digit indicates the pH, the second digit indicates the % H2O2, and the third digit indicates the incubation time (in hours). Thus, the first tube from left “604” had a pH of 6, no H2O2 included, and was incubated for 4 hours. The second tube from left “414” had a pH of 4, 1% H2O2, and was incubated for 4 hours. As shown, lowered pH slightly decolored the proteins solutions (compare the tubes at pH6, pH4, and pH3). However, the addition of 1% H2O2 to the pH-modified samples greatly improved decolorization. Protein denaturation at the lower pHs was not observed.

### Example 3: Decoloring Colored Protein Products Using Chemical Oxidation and Reduced pH

The methods of Example 2 were repeated with modification of the ultrafiltration concentrate to a pH of 3, pH of 4, or pH 6 and H2O2 concentrations of 0%, 1%, or 5%. Once the pH was adjusted and H2O2 was added, the samples were held for over 8 hours, e.g., 9 hours.

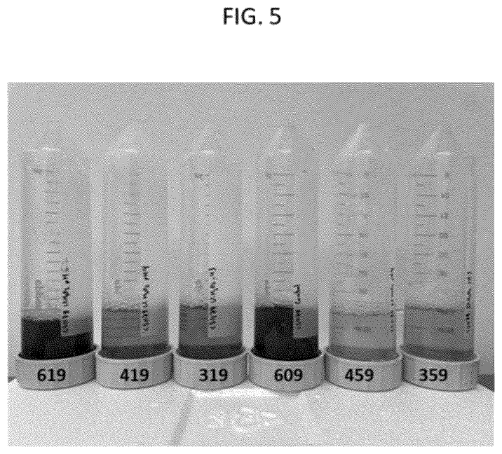

FIG. 5 shows illustrative samples of the treatments showing the effects of altered pH with or without a strong chemical oxidizer.

At pH6, the presence of 1% H2O2 had little observable effect on the protein concentrate's color. At pH4, 1% H2O2 for 9 hours improved the decolorating relative to the pH6 sample with 1% H2O2 and relative to a pH4, 1% H2O2 incubated for 4 hours (see FIG. 4). However, at pH4 with 9 hour incubations, samples incubated with 5% H2O2 had more decolorating than samples incubated with 1% H2O2. Like the data at pH4, samples at pH3, 1% H2O2 for 9 hours improved the decolorating relative to the pH6 sample with 1% H2O2 and relative to a pH3, 1% H2O2 incubated for 4 hours (see FIG. 4). However, at pH3 with 9-hour incubations, samples incubated with 5% H2O2 had more decolorating than samples incubated with 1% H2O2. The samples with 1% H2O2 at pH 3 or pH 4 had similar amounts of discoloration and the samples with 5% H2O2 at pH 3 or pH 4 has similar amounts of discoloration. Thus, reducing the pH from 6 to 4 or 3 improved decoloration. And, increasing the incubation time from four hours to nine hours also improved decoloration. Without wishing to be bound by theory, it is expected that incubations of twelve hours or more may reduce stability of a protein product. Protein denaturation at the lower pHs was not observed under the conditions of this example.

Although this example used ovalbumin as the tested protein, other protein concentrates, e.g., recombinant egg proteins, including ovomucoid, that have an undesirable coloration may be decolored by the methods described herein.

This example demonstrates the combination of the lower pH in the range of the pI of the protein along with a strong oxidizer will result in decoloring of the protein mixture from dark olive green to lighter yellow.

### Example 4: Decoloring Colored Protein Products Using Absorbents and Flocculants

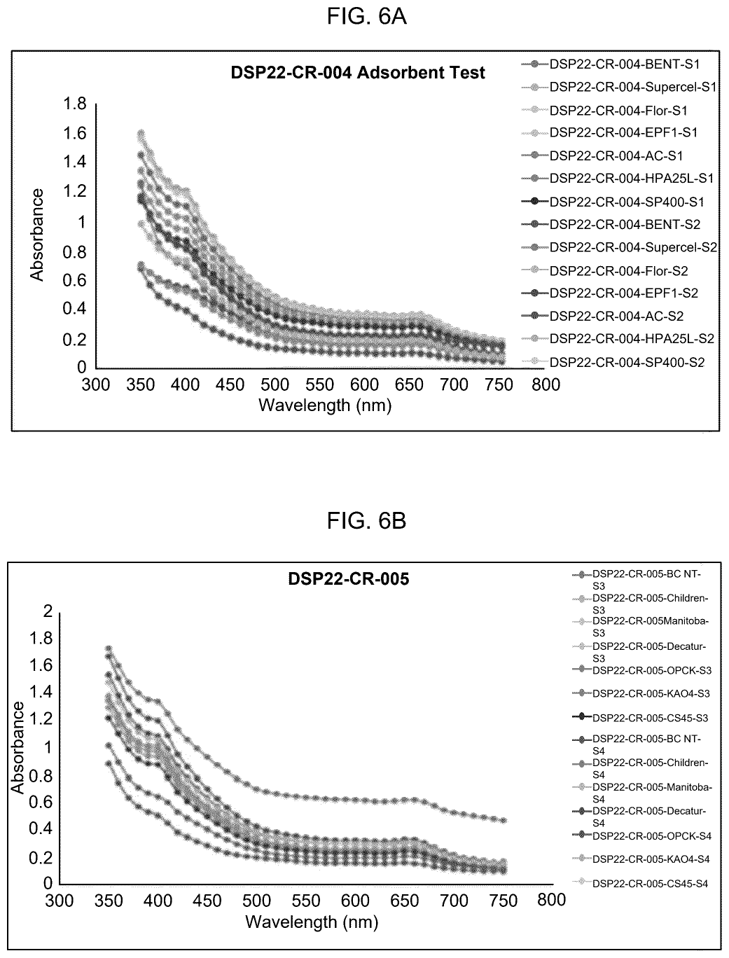

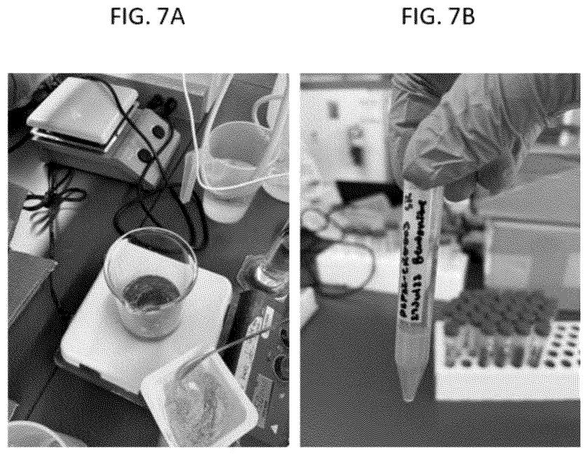

The protein ultrafiltration concentrate prepared as per Example 2 above was decolored using various adsorbents or flocculants. The procedure used was as follows: 1 g of the test adsorbent or flocculant was added to a 50 mL falcon tube, and 5 mL of 10% w/v protein ultrafiltration concentrate was added to the tube. The tubes were then placed on a roller shaker for 1 hour followed by centrifugation at 3214 g for 30 minutes. The absorbance of the supernatant was then measured. Each adsorbent or flocculant was tested twice. The list of adsorbents and flocculants tested were as follows: Bentonite, Activated Carbon, Diaion HPA25L, Relisorb SP400, EpMinerals FP1, Florisil, Celite Standard Supercel, Dicalite, Harborlite 800, Celite 545, Chitosan 85% Deacylated, Ultrapure Diatomaceous Earth, XAD4. The absorbance spectra between 350 and 750 nm is shown in FIG. 6A and FIG. 6B. In these figures, the lower a curve, the less absorbance of a treated protein ultrafiltration concentrate; with a lower absorbance meaning less color. Thus, in these figures, the lowest curves had the greatest decolorization. Bentonite showed the maximum color reduction (FIG. 7A and FIG. 7B).

The supernatant was further analyzed for the reduction of protein and the protein concentration reduction was within 10% of the initial concentration. Thus, use of various adsorbents or flocculants, e.g., bentonite, may be useful in that they can decolorize protein ultrafiltration concentrate without substantial loss of protein product. In some cases, activated carbon was found to decolorize protein ultrafiltration concentrates, however, there was not insignificant reduction of the protein product.

Although this example used ovalbumin as the tested protein, other protein concentrates, e.g., recombinant egg proteins, including ovomucoid, that have an undesirable coloration may be decolored by the methods described herein.

This example demonstrates various adsorbents or flocculants will result in decoloring of the protein mixture and without substantial loss of protein product.

### Example 5: Photochemical Processing of Colored Protein Products

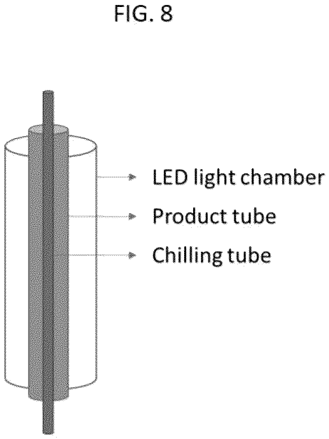

In this example, protein ultrafiltration concentrates, as obtained in Example 2, are decolorized using a device to photochemically oxidize the colored molecules. The design of an illustrative device is shown in FIG. 8. The protein ultrafiltration concentrate is pumped through a 1-inch tube containing an LED light to ensure the liquid is exposed to 1-2 kWhr/L photon intensity. The diameter of the tubing is limited to 1 inch to ensure uniform and complete light penetration. The product tube is further jacketed and cooled to maintain the temperature of the product at 10±° C.

1. A method for preparing a protein composition having a reduced quantity of a recombinant cell byproduct which provides an undesirable color to a protein composition, the method comprising steps of:
(a) obtaining a composition having an undesirable color and comprising a recombinant protein and a recombinant cell byproduct;
(b) processing the composition having the undesirable color under conditions that oxidizes the recombinant cell byproduct or under conditions that separate the recombinant protein and the recombinant cell byproduct; and
(c) collecting a composition comprising the oxidized recombinant cell byproduct or collecting the separated recombinant protein, thereby obtaining a protein composition having a reduced quantity of the recombinant cell byproduct which provides an undesirable color.

2. The method of claim 1, wherein the recombinant cell byproduct is an off-color component.

3. The method of claim 1, wherein the step of processing the composition comprises use of an absorbent or flocculant which captures, traps, or isolates the recombinant cell byproduct.

4. The method of claim 3, wherein the absorbent or the flocculant does not substantially capture, trap, or isolate the recombinant protein.

5. The method of claim 3, wherein the collecting the separated recombinant protein comprises use of gravity directed to the absorbent or the flocculant that has captured, trapped, or isolated the recombinant cell byproduct, comprise use of a filter that restricts passage of the absorbent or the flocculant that has captured, trapped, or isolated the recombinant cell byproduct and/or comprises use of centrifugation directed to the absorbent or the flocculant that has captured, trapped, or isolated the recombinant cell byproduct.

6. The method of claim 5, wherein when gravity or centrifugation is used, the collecting the separated recombinant protein comprises decanting or aspirating a protein composition having a reduced quantity of the recombinant cell byproduct which provides an undesirable color.

7. The method of claim 5, wherein when a filter is used, the collecting the separated recombinant protein comprises allowing the protein composition having a reduced quantity of the recombinant cell byproduct which provides an undesirable color to flow through the filter.

8. The method of claim 3, wherein the absorbent or the flocculant comprises one or more of Bentonite, Activated Carbon, Diaion HPA25L, Relisorb SP400, EpMinerals FP1, Florisil, Celite Standard Supercel, Dicalite, Harborlite 800, Celite 545, Chitosan 85% Deacylated, Ultrapure Diatomaceous Earth, XAD4.

9. (canceled)

10. The method of claim 3, wherein the absorbent or the flocculant comprises bentonite or activated carbon.

11. (canceled)

12. The method of claim 3, wherein the spectrophotometric absorbance of the obtained protein composition having a reduced quantity of the recombinant cell byproduct which provides an undesirable color is less than the spectrophotometric absorbance of the composition having an undesirable color and comprising a recombinant protein and a recombinant cell byproduct.

13. The method of claim 1, wherein the step of processing the composition comprises use of an oxidizing agent which oxidizes the recombinant cell byproduct, wherein the oxidizing agent is hydrogen peroxide.

14. (canceled)

15. The method of claim 13, wherein the step of processing the composition further comprises reducing the pH to: within a pH of 2 of the recombinant protein's pI; or about the pH of the recombinant protein's pI.

16. (canceled)

17. (canceled)

18. The method of claim 13, wherein the step of processing the composition further comprises adjusting the pH of the composition to between about a pH of 3 or 4.

19. The method of claim 13, wherein the step of processing the composition comprises an incubation period of about 9 hours or less than about 12 hours.

20. (canceled)

21. The method of claim 1, wherein the step of processing the composition comprises use of light which oxidizes the recombinant cell byproduct, and wherein the light is applied at an intensity and duration such that the recombinant cell byproduct is oxidized.

22. (canceled)

23. The method of claim 1, wherein processing the composition comprises agitation and occurs at room temperature or at a temperature less than room temperature.

24. (canceled)

25. (canceled)

26. The method of claim 1, wherein the composition comprising the recombinant protein and the recombinant cell byproduct was produced by fermentation of the recombinant cell.

27. The method of claim 1, wherein the composition comprising the recombinant protein and the recombinant cell byproduct was previously treated to remove: spent biomass including recombinant cells; protein molecules other than the recombinant protein; or small non-protein molecules.

28. (canceled)

29. (canceled)

30. The method of claim 27, wherein the treatment to remove small non-protein molecules comprises a diafiltration buffer and a step that concentrates the composition comprising the recombinant protein and the recombinant cell byproduct.

31. (canceled)

32. The method of claim 1, wherein the method further comprises a concentration step and/or diafiltration treatment of the separated recombinant protein to produce a protein-containing composition having a preferred pH and/or ionic condition.

33. The method of claim 32, wherein the protein-containing composition having a preferred pH and/or ionic conditions is further heat treated and/or dried, wherein the heat treatment and/or drying step produces a dry protein product having a reduced quantity of the off-color component.

34. (canceled)

35. The method of claim 1, wherein the protein product having a reduced quantity of the off-color component comprises an at least 50% reduction in off-color component quantity relative to the composition comprising a recombinant protein and a recombinant cell byproduct or an at least 75% reduction, at least 80% reduction, at least 90% reduction, or at least 95% reduction in the off-color component relative to the composition comprising a recombinant protein and a recombinant cell byproduct.

36. (canceled)

37. The method of claim 35, wherein less than about 5%, less than about 1%, less than about 0.1%, or less than about 0.01% of the weight of the protein product comprises the off-color component.

38. The method of claim 1, wherein the recombinant cell that expresses and/or secretes the recombinant protein and the recombinant cell byproduct is selected from a fungal cell, such as filamentous fungus or a yeast, a bacterial cell, a plant cell, an insect cell, or a mammalian cell.

39. The method of claim 1, wherein the recombinant cell that expresses and/or secretes the recombinant protein and the recombinant cell byproduct is selected from Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Pseudomonas spp., Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

40. The method of claim 38, wherein the recombinant cell that expresses and/or secretes the recombinant protein and the recombinant cell byproduct is a Pichia species.

41. The method of claim 40, wherein the Pichia species is Komagataella phaffii or Komagataella pastoris.

42. The method of claim 1, wherein the recombinant protein is an enzyme, a nutritive protein, a food ingredient, or a food additive.
